(19) United States
(12) Patent Application Publication
(10) Pub. No.: US 2022/0370073 A1
(43) Pub. Date: Nov. 24, 2022

(54) CARTRIDGE AND COUPLING MEMBER DETACHING METHOD

(71) Applicant: OLYMPUS CORPORATION, Tokyo (JP)

(72) Inventor: Tsutomu OKADA, Tachikawa-shi (JP)

(73) Assignee: OLYMPUS CORPORATION, Tokyo (JP)

(21) Appl. No.: 17/881,855

(22) Filed: Aug. 5, 2022

Related U.S. Application Data
(63) Continuation of application No. PCT/JP2020/004838, filed on Feb. 7, 2020.

(51) Int. Cl. A61B 17/122; A61B 17/10

(52) U.S. Cl. CPC A61B 17/1222; A61B 17/105

(57) ABSTRACT

A cartridge includes an insertion port into which at least a part of an applicator is insertable, a first region for accommodating a clip unit that can be releasably coupled to the applicator, and a second region for removing a coupling member from the applicator.

The present disclosure relates to a cartridge and a coupling member detaching method. This application is a continuation application based on PCT Patent Application No. PCT/JP2020/004838, filed Feb. 7, 2020, the content of which is incorporated herein by reference.

### BACKGROUND

In the related art, a ligature tool having a clip has been used by inserting into the body for ligating an opening formed in a tissue or a blood vessel. An endoscopic treatment tool is used when a tissue is ligated with a ligature tool. The ligature tool is coupled to an operation wire of the endoscopic treatment tool before being indwelled inside the body. After ligation of a tissue, a distal end part of the ligature tool is indwelled inside the body, and a proximal end part of the ligature tool retains a state of being coupled to the operation wire. For this reason, after indwelling of the ligature tool, there is a need to draw out the endoscopic treatment tool from the inside of the body and remove the proximal end portion of the ligature tool from the operation wire.

A method of removing a proximal end portion of a ligature tool from an operation wire, in which a user directly removes it by hand, and a method of using a cartridge are known. Since a ligature tool is a small component, it is necessary to pay close attention to removing work performed by hand. For this reason, a removing method using a cartridge is effective from the viewpoint of operability.

### SUMMARY

A cartridge according to an aspect includes an insertion port into which at least a part of an applicator is insertable, a first region for accommodating a clip unit that can be releasably coupled to the applicator, and a second region for removing a coupling member from the applicator.

The clip unit may have a clip arm configured to be opened and closed, and may include a second coupling member for coupling the clip unit with the applicator.

The second region of the cartridge may be able to receive the coupling member such that the coupling member is tilted at a detachment angle at which the coupling member is capable of being detached from the applicator.

The second region may be a cavity defined by an internal wall of the cartridge. The second region may communicate with the insertion port. The second region may allow the applicator and the coupling member inserted through the insertion port to be movable in the second region.

The cartridge may further include a detachment guide provided inside the cavity and capable of causing the coupling member to tilt at the detachment angle in response to abutment of the coupling member against the detachment guide.

A retraction restricting guide may be provided inside the cavity and may be capable of restricting retraction of the coupling member when the applicator is retracted.

The retraction restricting guide may be provided on a wall of the cavity and include an inclined surface inclined from a distal end portion of the wall toward the insertion port.

A detachment guide may be formed inside the cavity and extend from a first end in a distal direction so as to be curved in a direction intersecting an advance-retract direction of the applicator through the insertion port. A retraction restricting guide may be formed in the cavity. The retraction restricting guide may include an inclined surface inclined from a second end side of the detachment guide toward the insertion port and may restrict retraction of a distal end of the coupling member which is tilted at the detachment angle when the applicator is retracted. A distance between the first end of the detachment guide and a proximal end of the inclined surface of the retraction restricting guide may be shorter than a length of the coupling member which has been inserted into the cavity.

The coupling member may be engageable with a distal end portion of a wire of the applicator so as to be moveable in a direction intersecting a longitudinal axis of the wire. The coupling member may include an opening through which the wire can extend when the coupling member is tilted at the detachment angle and detached from the wire, or a distal end portion of the wire may include an opening through the coupling member can extend when the coupling member is tilted at the detachment angle and detached from the wire.

The clip unit may be capable of grasping a biological tissue. The second coupling member may be separated from the clip unit after the clip unit ligates the biological tissue.

The insertion port may include a first insertion port and a second insertion port. The first insertion port may communicate with the first region. The second insertion port may communicate with the second region.

The cartridge may further include a casing in which the insertion port is provided, and a slidable region in which the first region and the second region are provided.

A method of using a cartridge according to an aspect includes: inserting a coupling member and an applicator into a cartridge in a first state in which the coupling member is releasably connected to the applicator; shifting to a second state in which the coupling member is able to be detached from the applicator by advancing and retracting the applicator; and detaching the coupling member from the applicator by advancing and retracting the applicator in the second state. The method may further include connecting a second coupling member and the applicator by using the cartridge in a state in which the coupling member is detached from the applicator.

The coupling member may be detached from the applicator in a state in which the applicator is inserted into a first insertion port of the cartridge. The second coupling member may be connected to the applicator by retracting applicator from the first insertion port, and inserting the applicator into a second insertion port of the cartridge.

The method may further include: sliding a slider provided in the cartridge in a state in which the coupling member is detached from the applicator, and connecting the applicator to a second coupling member.

The coupling member may extend along a longitudinal axis of the applicator in the first state. The coupling member may extend in a direction intersecting the longitudinal axis in the second state.

The coupling member and the applicator may be shifted to the second state by advancing the applicator inside the cartridge such that the coupling member abuts against an inner wall of the cartridge.

The coupling member may be detached from the applicator by retracting the applicator inside the cartridge such that the coupling member in the second state may be caused to abut against the inner wall of the cartridge.

The applicator may include a wire and/or a sheath.

### BRIEF DESCRIPTION OF THE DRAWINGS

FIG. 1 is a perspective view illustrating an example of a ligature tool and an endoscopic treatment tool using a cartridge according to an exemplary embodiment.

FIG. 2 is a side view and partial cross-sectional view schematically illustrating the ligature tool using the cartridge according to the exemplary embodiment.

FIG. 3 is side view and partial cross-sectional view schematically illustrating the ligature tool using the cartridge according to the exemplary embodiment.

FIG. 4 is a cross-sectional view illustrating the cartridge according to the exemplary embodiment.

FIG. 5 is a partial cross-sectional view schematically illustrating a coupling member after ligation.

FIG. 6 is a cross-sectional view illustrating a usage state of the cartridge according to the exemplary embodiment.

FIG. 7 is a cross-sectional view illustrating a usage state of the cartridge according to the exemplary embodiment.

FIG. 8 is a cross-sectional view illustrating a usage state of the cartridge according to the exemplary embodiment.

FIG. 9 is a side view illustrating a proximal end portion of the coupling member.

FIG. 10 is a cross-sectional view illustrating a usage state of the cartridge according to the exemplary embodiment.

FIG. 11 is a cross-sectional view illustrating a cartridge according to an exemplary embodiment.

FIG. 12 is a cross-sectional view illustrating a usage state of the cartridge according to the exemplary embodiment.

FIG. 13 is a cross-sectional view illustrating a usage state of the cartridge according to the exemplary embodiment.

FIG. 14 is a cross-sectional view illustrating a usage state of a cartridge according to an exemplary embodiment.

FIG. 15 is a cross-sectional view illustrating a usage state of the cartridge according to the exemplary embodiment.

FIG. 16 is a cross-sectional view illustrating a cartridge according to an exemplary embodiment.

FIG. 17 is a cross-sectional view illustrating a usage state of the cartridge according to the exemplary embodiment.

FIG. 18 is a cross-sectional view illustrating a usage state of the cartridge according to the exemplary embodiment.

FIG. 19 is a top view illustrating a cartridge according to an exemplary embodiment.

FIG. 20 is another top view illustrating the cartridge according to the exemplary embodiment and is a partial cross-sectional view of a casing.

FIG. 21 is another top view illustrating the cartridge according to the exemplary embodiment and is a partial cross-sectional view of the casing.

FIG. 22 is a side view illustrating an exemplary embodiment of a connector of an operation wire and a proximal engagement portion of the coupling member.

### DETAILED DESCRIPTION OF THE PREFERRED EMBODIMENTS

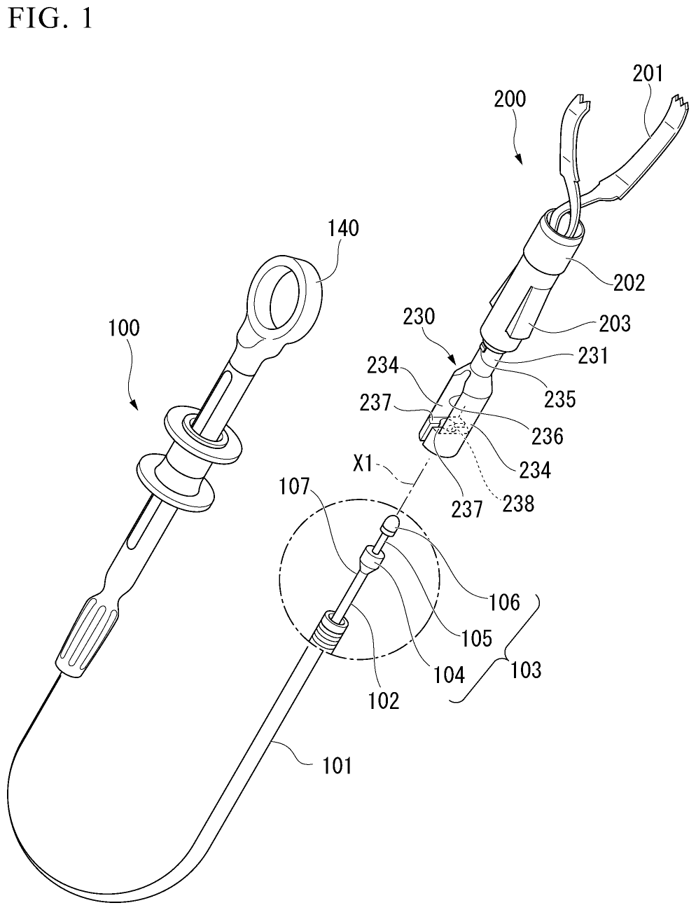

Hereinafter, a cartridge and a coupling member detaching method according to the present embodiment will be described with reference to FIGS. 1 to 10.

First, an endoscopic treatment tool 100 using a cartridge 1 according to the present embodiment will be described. For example, the endoscopic treatment tool 100 is a treatment tool for indwelling a ligature tool 200 having a clip main body 201 capable of grasping a biological tissue inside the body by using an endoscope.

The endoscopic treatment tool 100 includes an operation portion 140, a sheath 101, and an operation wire (wire) 102. The operation wire 102 is inserted into the sheath 101. In the following description, a centerline in a longitudinal direction in a state in which the operation wire 102 linearly extends will be referred to as a longitudinal axis X1. The operation portion 140 side of the endoscopic treatment tool 100 will be referred to as a proximal side, and a side on which the ligature tool 200 is attached will be referred to as a distal side. A proximal end portion of the operation wire 102 is coupled to the operation portion 140. A distal end portion of the operation wire 102 is configured to be retractable from a distal end of the sheath 101 by an advance-retract operation of the operation portion 140 with respect to the sheath 101. The operation wire 102 is formed using a metal single-strand wire or a twisted wire. The ligature tool 200 is detachably attached with respect to the distal end portion of the operation wire 102.

FIG. 1 illustrates the distal end portion of the operation wire 102 in an enlarged manner. A connector 103 is provided in the distal end portion of the operation wire 102. The connector 103 is a member coupled to a coupling member 230 of the ligature tool 200. The connector 103 has a large diameter portion 104, an arrowhead portion 106, and a shaft 105. The connector 103 has a constitution in which the large diameter portion 104 positioned in a proximal portion and the arrowhead portion 106 positioned in a distal portion are connected to each other by the shaft 105. The shaft 105 is a pillar member having a smaller diameter than the arrowhead portion 106.

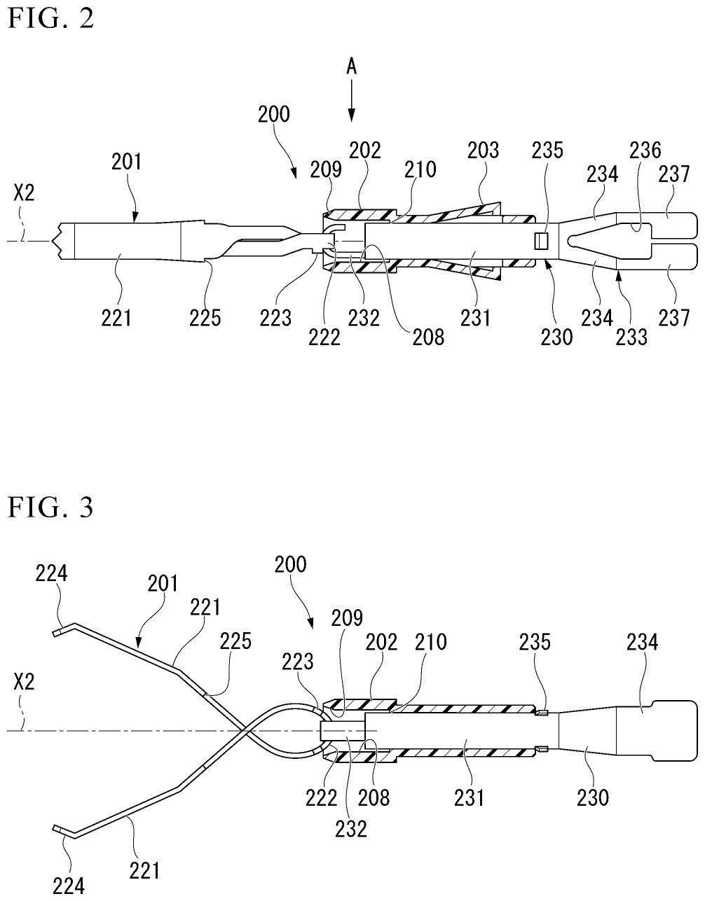

FIGS. 2 and 3 are views schematically illustrating the ligature tool 200 of the present embodiment and are partial cross-sectional views of the ligature tool 200 in the longitudinal axis X1 direction. FIG. 3 is a view in the arrow A direction indicated in FIG. 2. As illustrated in FIGS. 1 to 3, the ligature tool 200 includes the clip main body 201 (which will hereinafter be referred to as “a clip”), a holding tube 202, and the coupling member 230. The clip main body 201 side of the ligature tool 200 will be referred to as a distal side, and the coupling member 230 side of the ligature tool 200 will be referred to as a proximal side.

The clip main body 201 has a pair of arms 221 and a connection portion 222. For example, the clip main body 201 is formed by using a slender thin plate made of a metal such as a stainless steel, a cobalt-chromium alloy, or titanium. In the clip main body 201, the connection portion 222 subjected to bending in a thickness direction is formed in an intermediate portion of the slender thin plate, and both end portions of the plate form the pair of arms 221. The connection portion 222 is disposed on the proximal side of the pair of arms 221. The pair of arms 221 intersect each other on the distal side of the connection portion 222 and further extend toward a distal direction in a direction in which the arms 221 are separated from each other.

A pair of protrusion pieces 223 are formed in the connection portion 222. The pair of protrusion pieces 223 protrude outward in a direction orthogonal to the longitudinal direction in which the pair of arms 221 extend. The pair of protrusion pieces 223 may be formed at positions line-symmetrical with respect to a longitudinal axis X2 along the longitudinal direction.

Claw portions 224 are formed at end portions of the respective arms 221 on a side opposite to the connection portion 222. The claw portions 224 are bent in a direction so as to approach each other. The arms 221 have a bending tendency in a direction in which the arms 221 are separated from each other from the connection portion 222 side toward the claw portions 224 side.

As illustrated in FIG. 2, stepped portions 225 are formed at proximal parts of the respective arms 221 on the distal side of the intersecting part of the clip. The widths of the arms 221 change at the stepped portions 225 as boundaries. The arms 221 have width wider on the distal side than the stepped portions 225.

The holding tube 202 is a tubular member having a penetration path 208 penetrating the holding tube 202 in the longitudinal axis X2 direction. The penetration path 208 is a circular hole and penetrates the holding tube 202 from the distal end to a proximal end 41. The penetration path 208 has an inner diameter allowing at least a part of the proximal portion of the clip main body 201 to be inserted thereinto. A tapered portion 209 is formed in an inner wall of the distal end portion of the penetration path 208. The tapered portion 209 is formed to increase in diameter toward the distal end of the holding tube 202. A decreased diameter portion 210 having a smaller inner diameter than the distal side is formed in an intermediate region of the penetration path 208. A step is formed in the decreased diameter portion 210. The outer diameter of the holding tube 202 is larger than the inner diameter of the sheath 101 of the endoscopic treatment tool 100.

As illustrated in FIGS. 1 and 2, a pair of retractable wings 203 are provided in an outer circumferential portion of the holding tube 202. The retractable wings 203 are configured to be movable between in a state of protruding outward from the holding tube 202 and a state of being pressed and elastically deformed due to an external force and having entered the penetration path 208 side.

For example, the holding tube 202 is produced using a metal material such as a stainless steel, a titanium alloy (Ti-6AX4V or the like), or a cobalt-chromium alloy; or a highly rigid resin material such as a polyphthalamide PPA or a polyamide PA having a moderate elasticity.

The coupling member 230 is a member coupling the clip main body 201 and the operation wire 102 to each other. As illustrated in FIGS. 2 and 3, the coupling member 230 has a coupling main body 231, a hook portion 232, a proximal engagement portion 233, interlocking protrusions 235, and an opening 236. The coupling member 230 is molded using a resin material or the like having a predetermined strength.

The coupling main body 231 may only have a size which can be inserted into the penetration path 208 of the holding tube 202, and the shape thereof is not limited. The coupling main body 231 of the present embodiment has substantially a columnar shape, and the outer diameter of the coupling main body 231 is smaller than the inner diameter of the penetration path 208 of the holding tube 202. The hook portion 232 is provided at the distal end of the coupling main body 231, and the proximal engagement portion 233 is provided at the proximal end of the coupling main body 231.

The hook portion 232 is provided such that it protrudes in the distal direction from the distal end of the coupling main body 231. The hook portion 232 has a shape bent in a direction intersecting a central axis of the coupling main body 231 having a columnar shape. The hook portion 232 is configured to lock the connection portion 222 of the clip main body 201. The hook portion 232 is positioned on an inward side of an outer circumference of the coupling main body 231 when the coupling member 230 is viewed from the distal side and has a dimension which can be inserted into the penetration path 208 of the holding tube 202.

The proximal engagement portion 233 is configured to engage with the connector 103 of the operation wire 102. The proximal engagement portion 233 extends in a proximal direction of the coupling main body 231. As illustrated in FIG. 2, the proximal engagement portion 233 has a pair of legs 234 and a pair of proximal interlocking ends 237. The pair of legs 234 extend from the coupling main body 231 to the proximal side, and the opening 236 is formed between the distal portions of the pair of legs 234. The opening 236 opens on a lateral side of the proximal engagement portion 233.

The proximal interlocking ends 237 are formed to protrude toward the central axis of the coupling member 230 such that a separation distance between the proximal ends of the pair of legs 234 is narrowed. The opening width of the opening 236 on the proximal side is narrowed by the proximal interlocking ends 237. As illustrated in FIG. 1, recessed portions 238 having an arc shape are respectively formed on facing surfaces of the pair of proximal interlocking ends 237. When an external force is applied to the legs 234, the legs 234 are elastically deformed to an outward side of the coupling member 230 in the radial direction, and the pair of proximal interlocking ends 237 are separated from each other or approach each other. The opening 236 has a size allowing the arrowhead portion 106 of the connector 103 to move therethrough.

The interlocking protrusions 235 are provided on an outer circumferential surface of the proximal portion of the coupling main body 231, and protrude in the radial direction. As illustrated in FIG. 3, two interlocking protrusions 235 are provided at symmetrical positions with the central axis of the coupling main body 231 sandwiched therebetween. The two interlocking protrusions 235 protrude to the extent that they cannot enter the penetration path 208 of the holding tube 202. The interlocking protrusions 235 restrict advancing of the coupling member 230 with respect to the holding tube 202. When the coupling member 230 is inserted into the penetration path 208 of the holding tube 202 and is advanced, the interlocking protrusions 235 abut the proximal end 41 of the holding tube 202 and restrict additional advance of the coupling member 230.

As illustrated in FIGS. 1 to 3, in the ligature tool 200, the coupling main body 231 of the coupling member 230 is inserted into the penetration path 208 of the holding tube 202 in a state in which the connection portion 222 of the clip main body 201 is locked with the hook portion 232. The clip main body 201 is disposed on the distal side of the holding tube 202 along the longitudinal axis X2 of the holding tube 202. The proximal engagement portion 233 of the coupling member 230 is exposed on the proximal side of the holding tube 202.

The coupling member 230 and the clip main body 201 are provided to be capable of advancing and retracting in the longitudinal axis X2 direction with respect to the holding tube 202. The advance of the coupling member 230 with respect to the holding tube 202 is restricted by the interlocking protrusion 235. When the coupling member 230 advances to a most forward position, an locking part between the hook portion 232 and the connection portion 222 is positioned inside the penetration path 208, and thus a state in which the clip main body 201 is locked with the hook portion 232 is retained. The clip main body 201 and the coupling member 230 are configured to retract to positions where the stepped portions 225 of the clip main body 201 abut the decreased diameter portion 210 of the holding tube 202.

The cartridge 1 will be described. The cartridge 1 is a cartridge for removing the coupling member 230 which is separated from the ligature tool 200 and has been engaged with the operation wire 102 after the ligature tool 200 is indwelled inside the body.

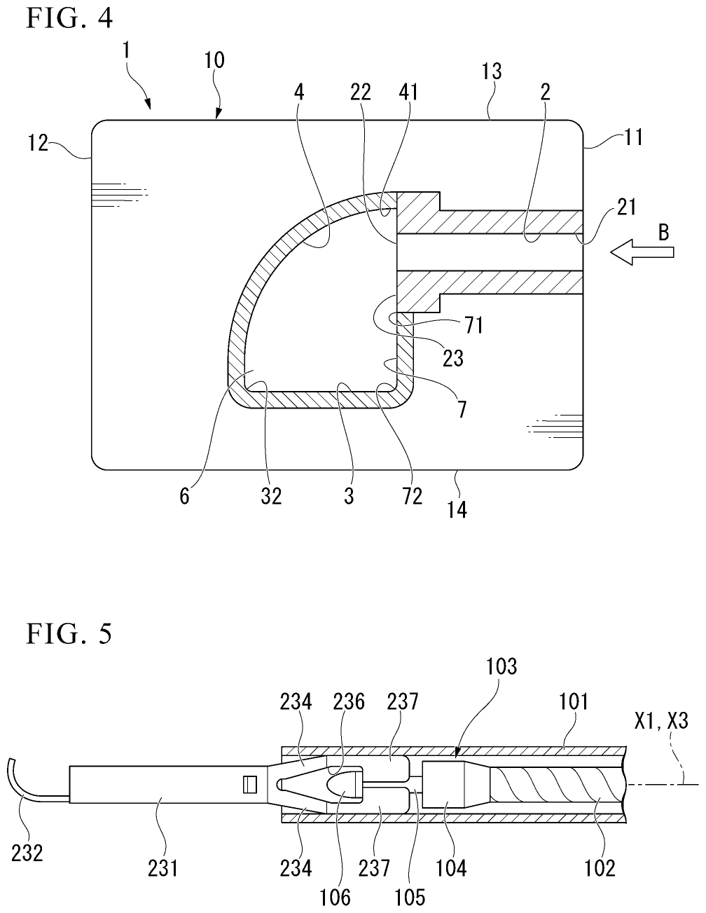

In the cartridge 1, an insertion path 2 and a cavity 6 are formed in a casing 10. For example, the casing 10 has a three-dimensional shape having a hollow portion. Regarding the cartridge 1, an example in which a wall portion forming the cavity 6 is fixed inside the hollow portion of the casing 10 or an example in which a wall portion forming the cavity 6 is sandwiched and fixed between two flat plates can be adopted. The casing 10 has a size easy for a user to hold by hand. In the examples illustrated in FIGS. 4 to 11, the cartridge 1 is illustrated in a cross-sectional view in a direction in which the operation wire 102 and the sheath 101 are inserted (a direction indicated by the arrow B in FIG. 4).

As illustrated in FIG. 4, an insertion port 21 is formed in the end portion of the casing 10. The cavity 6 is formed inside the casing 10. The cavity 6 is a space regulated by walls 3, 4, and 7 provided inside the casing 10. The cavity 6 is formed to communicate with the insertion port 21. In the present embodiment, the insertion path 2 is formed between the cavity 6 and the insertion port 21. The insertion path 2 has a size allowing the sheath 101 of the endoscopic treatment tool 100 to be inserted thereinto. The insertion path 2 is a lumen having a size allowing the sheath 101 to advance and retract along the longitudinal axis X1 of the sheath 101. The cavity 6 and the insertion port 21 communicate with the insertion path 2.

Hereinafter, in the description of the cartridge 1, an end portion in which the insertion port 21 is formed will be referred to as a proximal end 11, and an end portion of the cartridge 1 on a side opposite to the proximal end 11 in the arrow B direction (advance-retract direction) will be referred to as a distal end 12. When viewed in an entry direction (arrow B direction) of the operation wire 102 from the proximal end 11 of the cartridge 1, an end portion of the cartridge 1 on the right side (an end portion on the upper side in FIG. 4) will be referred to as a first side end 13, and an end portion thereof on the left side (an end portion on the lower side in FIG. 4) will be referred to as a second side end 14.

The cavity 6 is formed on the distal side of a distal opening 22 of the distal end of the insertion path 2. In the present embodiment, the cavity 6 is surrounded and regulated by the first wall 4, the second wall 3, and the third wall 7. The third wall 7 is substantially orthogonal to the entry direction of the operation wire 102 and is formed on the same plane as the distal opening 22. The third wall 7 extends from an end portion 71 of the distal opening 22 on the second side end 14 side toward the second side end 14. The second wall 3 extends from the end portion 71 of the third wall 7 on the second side end 14 side toward a distal end 32 in a direction substantially orthogonal to the third wall 7. The second wall 3 is substantially parallel to the entry direction of the operation wire 102. The first wall 4 is a wall extended to be inclined from the proximal end 41 on the first side end 13 side of the distal opening 22 to the distal end 32 of the second wall 3. In the example illustrated in FIG. 4, the first wall 4 has a curved shape projecting toward an outside of the casing 10. The cavity 6 has a sector shape in which a central angle is substantially a right angle. The first wall 4 is a wall separated from the insertion port 21 to the distal side and facing the insertion port 21 on an extended line of the distal opening 22. The first wall 4 is an example of a detachment guide which will be described below.

The cavity 6 has a space in which the operation wire 102 is capable of advancing and retracting and the coupling member 230 can be tilted at a detachment angle at which the coupling member 230 can be detached from the wire. Details will be described below. The cavity 6 functions as an accommodation chamber in which the coupling member 230 removed from the wire can be accommodated.

A method of attaching the ligature tool 200 to an endoscopic treatment tool and a method of using the ligature tool 200 will be described. Since the ligature tool 200 is a small-sized treatment tool, a known attachment cartridge is used when the ligature tool 200 is attached to the endoscopic treatment tool. The ligature tool 200 is housed inside the attachment cartridge, and an insertion opening for the operation wire 102 is formed on the proximal engagement portion 233 side of the coupling member 230. A user inserts the connector 103 of the operation wire 102 into the insertion opening while the user grasping the attachment cartridge, and the connector 103 is caused to approach the proximal engagement portion 233 of the ligature tool 200 from the proximal side. When the arrowhead portion 106 is caused to abut the proximal ends of proximal interlocking ends 237, the user further pushes the operation wire 102, and thus a force is applied to the legs 234, and the legs 234 are elastically deformed in a direction in which they are separated from each other. The arrowhead portion 106 enters a gap between the proximal interlocking ends 237 generated as a result of elastic deformation. The gap between the recessed portions 238 of the pair of proximal interlocking ends 237 is smaller than the diameter of the arrowhead portion 106 and is substantially equivalent to the diameter of the shaft 105. Therefore, when the arrowhead portion 106 enters the distal side from the proximal interlocking ends 237, the external force to the legs 234 is released, and the legs 234 return to original positions. The arrowhead portion 106 is disposed between the legs 234, the shaft 105 is clamped by the proximal interlocking ends 237, and the coupling member 230 is engaged with the connector 103. At this time, the clip main body 201, the holding tube 202, and the coupling member 230 are in a state disposed along the axis of the operation wire 102, and the state is retained by the attachment cartridge.

The endoscopic treatment tool 100 is inserted into an endoscope insertion portion, and the ligature tool 200 is indwelled inside the body. Before the endoscopic treatment tool 100 is inserted into the endoscope insertion portion, the attachment cartridge is removed. When at least a part of the attachment cartridge is opened in a direction intersecting the longitudinal axis X2 of the holding tube 202, the ligature tool 200 can be taken out from the attachment cartridge. After this, the proximal ends of the retractable wings 203 of the holding tube 202 are abutted the tip of the sheath 101 by retracting the operation wire 102. In the state, the endoscopic treatment tool 100 is inserted into the endoscope insertion portion.

When a tissue is sandwiched between the claw portions 224 of the clip main body 201, the clip main body 201 is retracted into the holding tube 202, and the proximal portion of the clip main body 201 is accommodated, the pair of arms 221 of the clip main body 201 approach each other, and the ligature tool 200 is retained a state in which the tissue is clamped therebetween.

The ligature tool 200 of the present embodiment is a ligature tool 200 which is configured to be regrasped and is configured to advance and retract the clip main body 201 with respect to the holding tube 202 at the time of indwelling. When the clip main body 201 is at a position where it has advanced with respect to the holding tube 202, the pair of arms 221 are in a separated state. In the ligature tool 200 of the present embodiment, the arms 221 intersect each other, and a loop of the connection portion 222 is larger than the opening of the penetration path 208 of the holding tube 202. As a result, when the connection portion 222 retracts while abutting the tapered portion 209 of the holding tube 202, the pair of claw portions 224 are further separated from each other than the state illustrated in FIG. 3. When the operation wire 102 is retracted while the claw portions 224 are pressed against a tissue (ligation object) in this state, the pair of arms 221 approach each other while the claw portions 224 are pressed against the tissue, and thus the tissue is sandwiched therebetween. The clip main body 201 is configured to advance and retract with respect to the holding tube 202 to a position where stepped portions 225 of the clip main body 201 abut the decreased diameter portion 210. For this reason, for example, when the clip main body 201 is advanced again after a tissue is temporarily grasped, the grasped tissue is released and the tissue can be grasped again. If a tissue can be grasped by the clip main body 201 in a desired state, the operation wire 102 is further retracted from a state in which the stepped portions 225 of the clip main body 201 have abutted the decreased diameter portion 210. Consequently, since the stepped portions 225 have abutted the decreased diameter portion 210, the clip main body 201 does not retract, but a force is applied to the coupling member 230 in a retraction direction. Since the proximal engagement portion 233 is housed inside the sheath 101, it is not elastically deformed to the outward in the radial direction, and the proximal interlocking ends 237 are retained in a closely disposed state. As a result, as illustrated in FIG. 5, the hook portion 232 is deformed, and the hook portion 232 is detached from the connection portion 222. When the hook portion 232 is detached from the connection portion 222, the coupling member 230 comes out from the holding tube 202, and the ligature tool 200 is indwelled inside the body. After indwelling, the coupling member 230 is removed from the inside of the body in a state of being engaged with the connector 103.

Next, a method of removing the coupling member 230 of the ligature tool 200 from the operation wire 102 will be described. As illustrated in FIG. 5, in the coupling member 230 which has been removed from the inside of the body, the proximal engagement portion 233 is accommodated inside the sheath 101. The coupling member 230 engaged with the operation wire 102, and distal end parts of the operation wire 102 and the sheath 101 are inserted into the insertion port 21 of the cartridge 1. The insertion path 2 is provided such that the sheath 101 is capable of advancing and retracting along the longitudinal axis X1. For this reason, the sheath 101 which has been inserted into the insertion path 2 advances along the longitudinal axis X1.

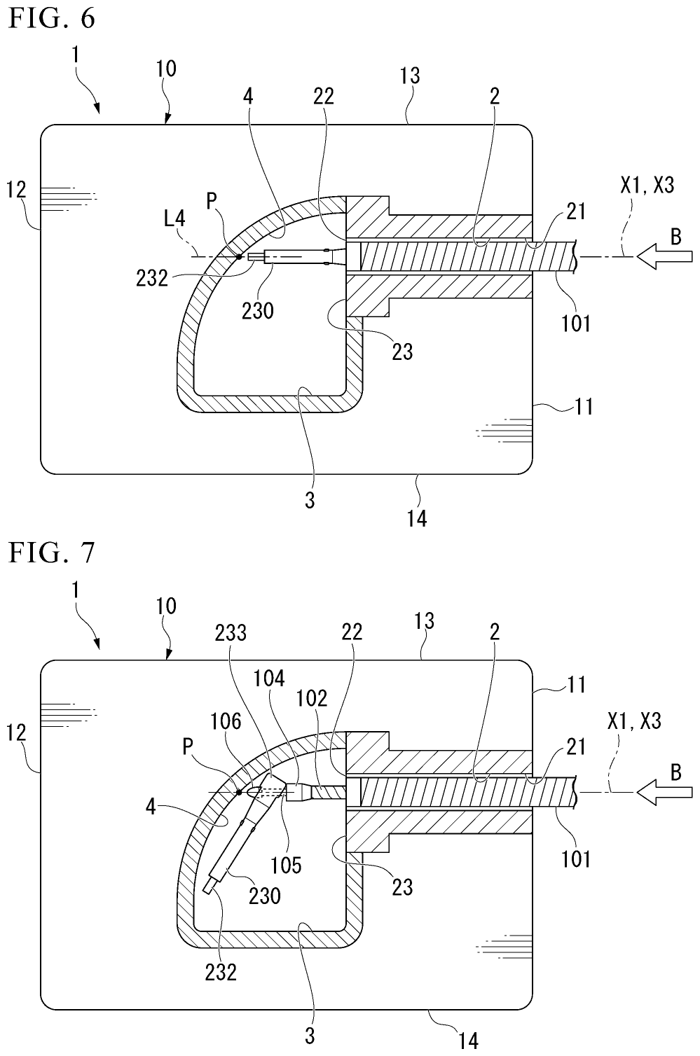

As illustrated in FIG. 6, when the distal end of the sheath 101 is inserted into to the distal opening 22, the coupling member 230 enters the inside of the cavity 6 along the longitudinal axis X2, and the hook portion 232 is disposed close to the first wall 4. Thereafter, when a user advances the operation wire 102 by operating the operation portion 140, the coupling member 230 is entirely exposed from the sheath 101, and the hook portion 232 (distal end of the coupling member 230) abuts the first wall 4. Although it is not illustrated, a stopper such as a projection for restricting advance of the distal end of the sheath 101 may be provided in the distal opening 22 in order for a user easily recognizes that the tip of the sheath 101 has advanced to the distal opening 22.

When the operation wire 102 is advanced in a state in which the distal end of the coupling member 230 abuts the first wall 4, the arrowhead portion 106 advances with respect to the coupling member 230 inside the opening 236. Since the opening 236 which is a gap between the legs 234 opens larger than the size of the arrowhead portion 106, when a proximal end of the arrowhead portion 106 is separated to a side in front of the proximal interlocking ends 237, restriction on the coupling member 230 in a direction along the longitudinal axis X1 of the operation wire 102 is canceled. As a result, the coupling member 230 is in a state to be capable of being inclined with respect to the longitudinal axis X1.

The first wall 4 intersects the longitudinal axis X2 of the insertion path 2, that is, the longitudinal axis X1 of the operation wire 102. For this reason, as illustrated in FIG. 7, in accordance with advance of the operation wire 102, the distal end of the coupling member 230 moves to the second wall 3 side along the first wall 4. At this time, the coupling member 230 which has been able to be inclined is inclined with respect to the operation wire 102. The operation wire 102 advances along the longitudinal axis X1, and the relative position of the arrowhead portion 106 with respect to the coupling member 230 moves to the position indicated by the reference sign b in FIG. 9.

In the present embodiment, since the first wall 4 has an arc shape, the distal end of the coupling member 230 which has abutted the first wall 4 smoothly moves to the second side end 14 side, and thus the coupling member 230 is caused to be tilted with respect to the operation wire 102.

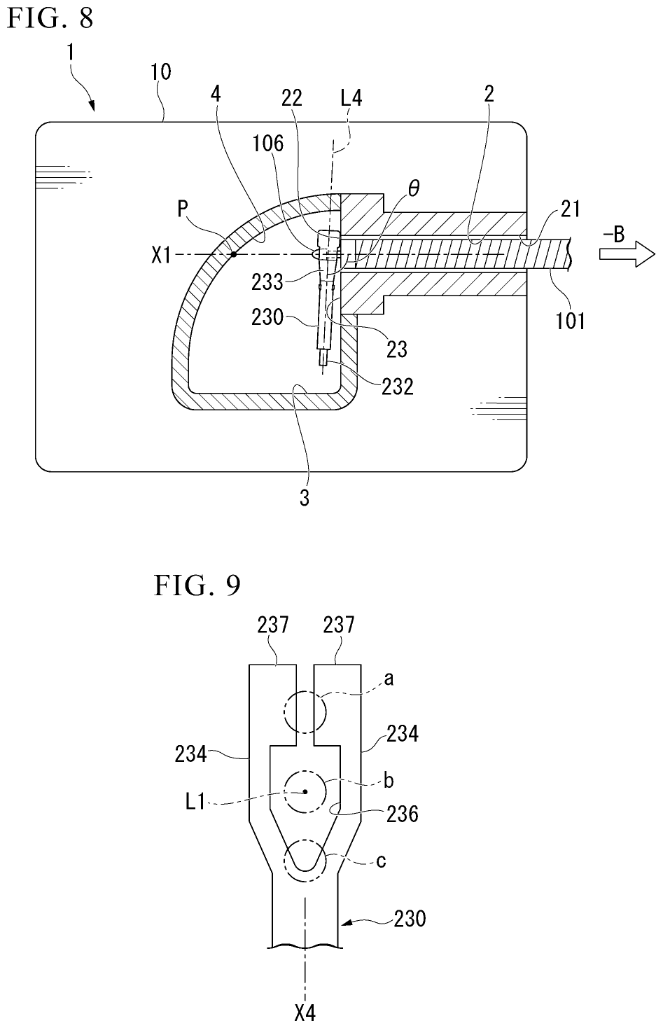

An angle at which the coupling member 230 can be detached from the operation wire 102 will be referred to as a detachment angle. The detachment angle is an angle of the coupling member 230 with respect to the operation wire 102 in a state in which the arrowhead portion 106 is positioned inside the opening 236 of the coupling member 230 and the arrowhead portion 106 comes out to a lateral side of the coupling member 230 through the opening 236, and an engagement between the operation wire 102 and the coupling member 230 becomes to be releasable. FIG. 9 is a schematic view of the state in FIG. 8 viewed from the distal end side of the arrowhead portion 106. As illustrated in FIG. 8, when the longitudinal axis X2 of the coupling member 230 tilts with respect to the operation wire 102, and when the arrowhead portion 106 is at the position indicated by the reference sign b in FIG. 9 and the longitudinal axis X1 of the operation wire 102 is at a position intersecting the opening 236, the arrowhead portion 106 is allowed to move to a lateral side inside the opening 236. The detachment angle is determined based on the shape or the like of the opening of the coupling member 230. For example, the coupling member 230 need only tilt such that the opening 236 opens along the longitudinal axis X1 of the operation wire 102.

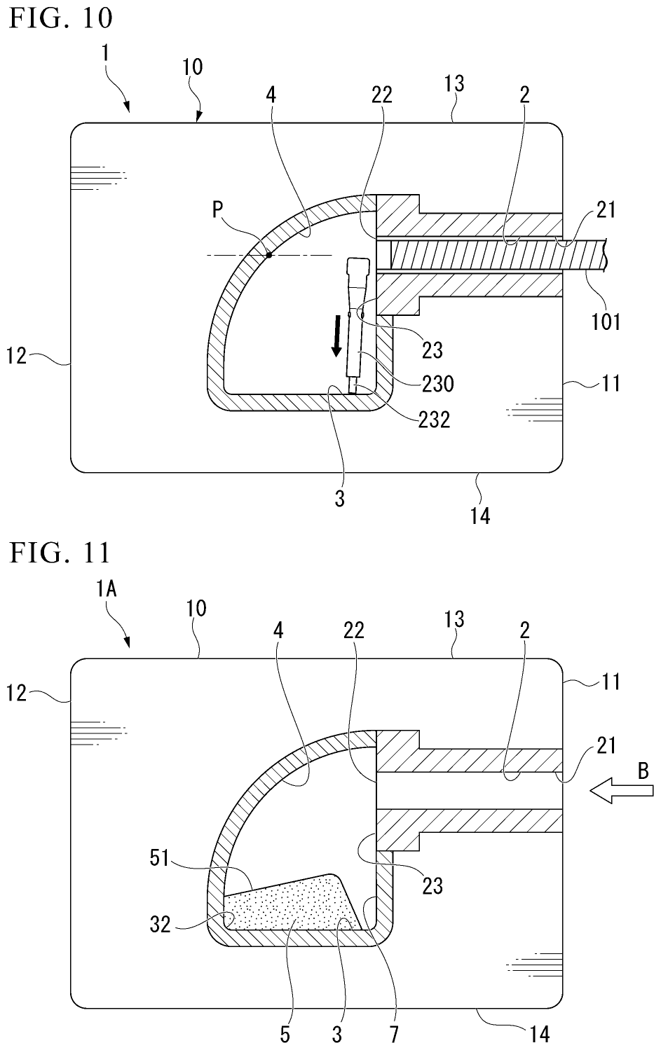

When the operation wire 102 is retracted with respect to the sheath 101 after the coupling member 230 tilts at the detachment angle, the arrowhead portion 106 positioned at the reference sign b in FIG. 9 comes out from the opening 236, and as illustrated in FIG. 10, the coupling member 230 falls off from the operation wire 102. As illustrated in FIG. 8, when the operation wire 102 is retracted, a lateral side of the proximal engagement portion 233 of the coupling member 230 abuts the distal end of the sheath 101 or an outer edge portion 23 of the distal opening 22, the arrowhead portion 106 smoothly comes out from the coupling member 230 by only a retraction operation of the operation wire 102. The outer edge portion 23 of the distal opening 22 functions as a detachment guide. The outer edge portion 23 of the distal opening 22 constitutes a part of the third wall 7. The fallen coupling member 230 is accommodated inside the cavity 6.

When a user inserts the sheath 101 into the insertion port 21 of the cartridge 1 and advances the operation wire 102, and then retracts the operation wire 102, the coupling member 230 easily falls off from the operation wire 102. Namely, after the sheath 101 is inserted into the cartridge 1, a user can remove the coupling member 230 from the operation wire 102 by a simple operation advancing and retracting the operation wire 102.

Since the separated coupling member 230 is a small component, a manual disengagement operation requires detailed work. In contrast, in the cartridge 1 according to the present embodiment, the coupling member 230 can be removed from the operation wire 102 by inserting the sheath 101 and only performing a simple operation of advancing and retracting the operation wire 102.

The cartridge is not limited to the configuration disclosed in the foregoing embodiment. For example, as in a cartridge 1A of a first modified example of the exemplary embodiment illustrated in FIGS. 11 to 13, a retraction restricting guide 5 may be provided inside the cavity 6. The retraction restricting guide 5 is a guide for restricting retraction of the distal end of the coupling member tilting at the detachment angle. For example, the retraction restricting guide 5 has a constitution having an inclined surface 51 inclined from the distal end side toward the distal opening 22 side on the second wall 3 side of the cavity 6.

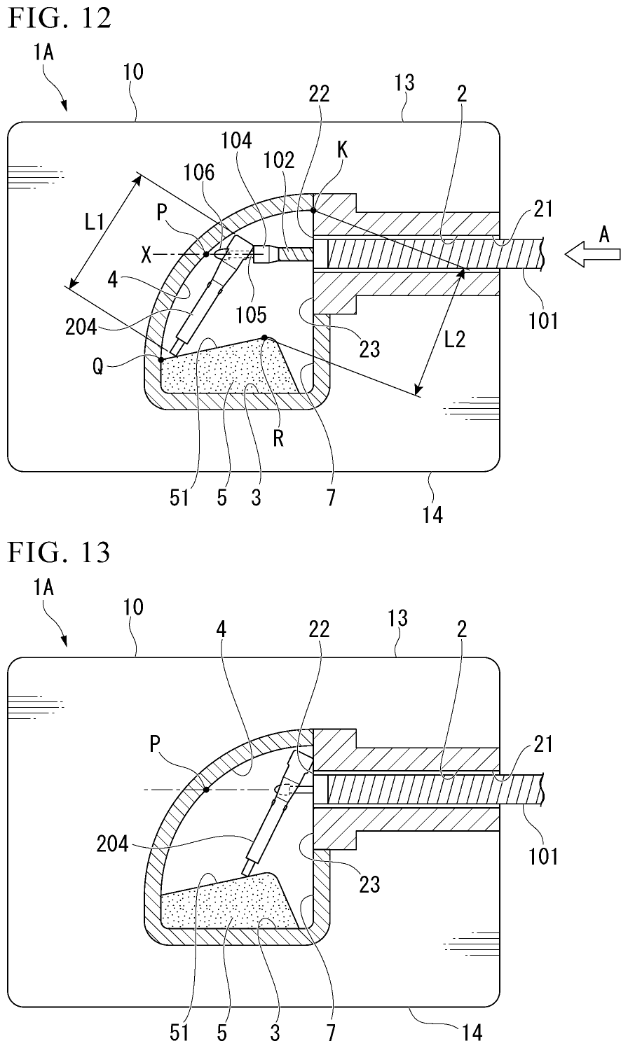

For example, the retraction restricting guide 5 may include the inclined surface 51 described below. As illustrated in FIG. 12, an intersection between the end portion of the retraction restricting guide 5 on the distal side and the first wall 4 is called an intersection Q, and an intersection point where the centerline of the insertion path 2, that is, an extended line along the longitudinal axis X1 of the operation wire 102 intersects the first wall 4, is called a point P. A distance between the intersection Q and the point P is shorter than a length L1 of the coupling member 230 in the longitudinal axis X2 direction. The inclined surface 51 is inclined as approaching the distal opening 22 from the intersection Q toward the proximal side. The angle between the inclined surface 51 and the first wall 4 at the intersection Q is smaller than the right angle.

When the operation wire 102 is further advanced after the hook portion 232 has abutted the first wall 4, the coupling member 230 tilts with respect to the longitudinal axis X1 of the operation wire 102. At this time, the arrowhead portion 106 protrudes from the opening 236 to a lateral side of the coupling member 230, and the arrowhead portion 106 relatively moves to the position indicated by the reference sign a in FIG. 9. Further, when the operation wire 102 advances, the hook portion 232 of the distal end of the coupling member 230 which has abutted the first wall 4 moves to the intersection Q along the first wall 4. At this time, the arrowhead portion 106 relatively moves from the position of the reference sign a illustrated in FIG. 9 to the position of the reference sign b. Thereafter, in accordance with retraction of the operation wire 102, the coupling member 230 retracts while the hook portion 232 comes into contact with the inclined surface 51. As a result, the hook portion 232 is positioned in front of the proximal end portions 237 and comes into contact with the inclined surface 51, and the coupling member 230 retracts while a state of obliquely tilting with respect to the operation wire 102 is retained. At a rear end R of the inclined surface 51, while the arrowhead portion 106 is kept at the position indicated by the reference sign b in FIG. 9, the coupling member 230 moves toward the first side end 13. A distance L2 between the rear end R and an intersection K between the proximal end 41 of the first wall 4 and the third wall 7 is shorter than the length L1 of the coupling member 230. As a result, as illustrated in FIG. 13, when the coupling member 230 retracts to a most retracted position after retraction of the operation wire 102, the proximal interlocking ends 237 of the coupling member 230 come into contact with the third wall 7 in the vicinity of the intersection K, and the hook portion 232 is positioned on the distal side of the rear end R of the inclined surface 51. When the operation wire 102 is operated to retract in this state, retraction of the hook portion 232 is restricted, and the proximal interlocking ends 237 abut the third wall 7 so that retraction of the proximal interlocking ends 237 is restricted. Therefore, as illustrated in FIG. 13, the coupling member 230 retains a tilted state. When the operation wire 102 retracts while the tilted state of the coupling member 230 is retained, the arrowhead portion 106 is smoothly detached from the opening 236.

As in the first modified example, since the retraction restricting guide 5 is provided, the operation wire 102 is capable of coming out from the coupling member 230 while retraction of the distal end part of the coupling member 230 is restricted. As a result, the operation wire 102 is easily drawn out from the coupling member 230.

Since the retraction restricting guide 5 has the inclined surface 51, the coupling member 230 can be guided such that the relative positions of the proximal interlocking ends 237 with respect to the arrowhead portion 106 become favorable positions.

The retraction restricting guide 5 is not limited to the first modified example and need only be constituted to restrict retraction of the distal end of the coupling member 230. For example, as shown in FIG. 12, a projection protruding from an inner surface of the casing 10 may be provided at a position of the point R between the second wall 3 and the distal opening 22. As a result, the distal end of the coupling member 230 comes into contact with the projection so that retraction of the coupling member 230 is restricted at the time of retraction of the operation wire 102, and the operation wire 102 is retracted while movement of the proximal interlocking ends 237 and the hook portion 232 of the coupling member 230 is restricted.

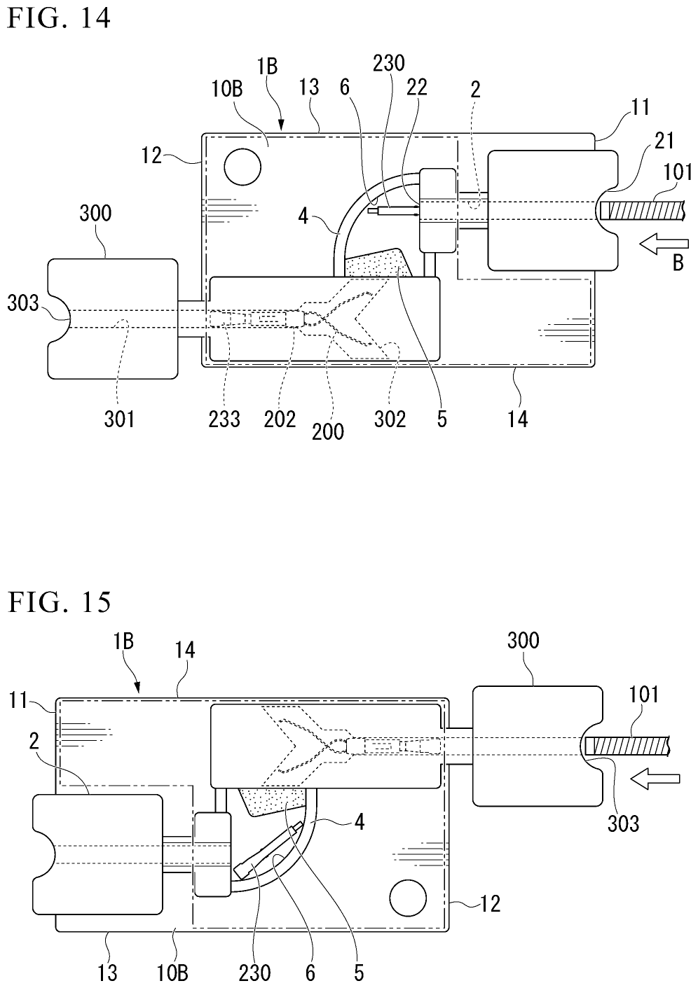

Other modified examples of the cartridge are illustrated in FIGS. 14 to 21. A cartridge 1B according to a second modified example of the exemplary embodiment illustrated in FIGS. 14 and 15 is an example in which the attachment cartridge of the ligature tool 200 and a detachment cartridge of the coupling member 230 are integrally provided. As illustrated in FIGS. 14 and 15, in the cartridge 1B according to the present modified example, a mechanism of the detachment cartridge of the coupling member 230 is provided at the proximal end 11 of a casing 10B, and a mechanism of the attachment cartridge of the ligature tool 200 is provided at the distal end 12. The detachment cartridge of the coupling member 230 has a constitution similar to that of the cartridge 1A illustrated in FIGS. 12 and 13. An accommodation portion 302 of the ligature tool 200 is provided between the cavity 6 and the second side end 14 of the casing 10B. The ligature tool 200 is accommodated inside the accommodation portion 302. A sheath insertion portion 303 of the attachment cartridge is provided such that it protrudes outward beyond the distal end 12 of the casing 10B. When the coupling member 230 is removed, as illustrated in FIG. 14, the sheath 101 is inserted through the insertion port 21 of the casing 10B, and an operation of removing the coupling member 230 described above is performed. When the ligature tool 200 is attached, as illustrated in FIG. 15, the sheath 101 is inserted into the sheath insertion portion 303 of the casing 10B on the distal end 12 side, and the connector 103 is engaged with the proximal interlocking ends 237 by advancing the operation wire 102. When the endoscopic treatment tool 100 is retracted after the operation wire 102 and the ligature tool 200 are engaged with each other, the ligature tool 200 becomes in a usable state.

According to the cartridge 1B of the present modified example, work of removing the coupling member 230 from the operation wire 102 and work of attaching a new ligature tool 200 to the operation wire 102 can be continuously and smoothly performed with one cartridge 1B. The order of the work of removing the coupling member 230 from the operation wire using the cartridge 1B and the work of attaching the ligature tool 200 to the operation wire 102 is not particularly limited.

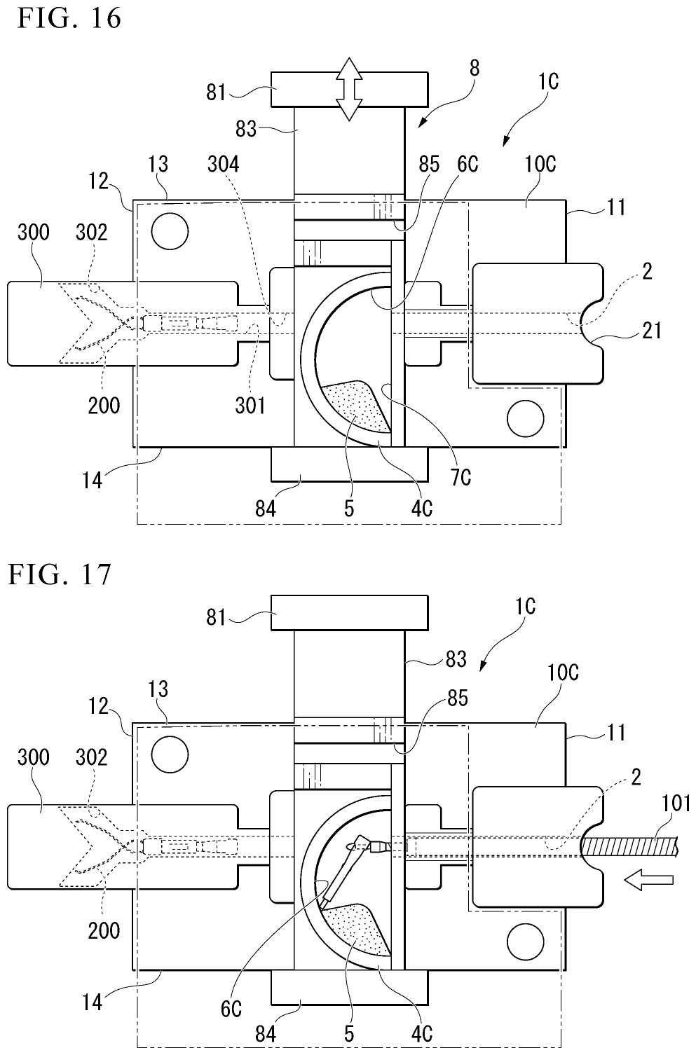

FIGS. 16 to 18 illustrate a cartridge 1C according to a third modified example of the exemplary embodiment. In the cartridge 1C according to the present modified example, similar to the second modified example, the detachment cartridge of the coupling member 230 and the attachment cartridge of the ligature tool 200 are integrally provided. In the cartridge 1C according to the present modified example, an insertion port of the sheath 101 is an example. The insertion port 21 is formed at the proximal end 11 of a casing 10C, a cavity 6C is disposed in a central portion, and the ligature tool 200 is accommodated in the distal end portion.

As illustrated in FIG. 16, the cavity 6C is regulated by a first wall 4C constituted of a semi-circular curved portion and a third wall 7C on the proximal side. A slide mechanism 8 of the first wall 4C is provided in the central portion of the casing 10C. The slide mechanism 8 is a mechanism for sliding movement of the first wall 4C regulating the cavity 6C in a direction orthogonal to the insertion path 2. The slide mechanism 8 has handles 81 and 84, a bar 83, and a groove 82. The first handle 81 and the second handle 84 are provided in both end portions of the bar 83. The first wall 4C is fixed to the second handle 84 side of the bar 83, and a sheath insertion path 85 is formed between the first wall 4C and the first handle 81. The bar 83 and the first wall 4C are attached to the casing 10C such that they can slide inside the groove 82.

In the casing 10C, the insertion port 21 and the insertion path 2 are formed on the proximal end side of the slide mechanism 8. The accommodation portion 302 of the ligature tool 200 is provided on the distal side from the slide mechanism 8 in the casing 10C. The ligature tool 200 is accommodated inside the accommodation portion 302. Each of the distal end of the insertion path 2 and a proximal opening 304 of the accommodation portion 302 communicates with the groove 82.

As illustrated in FIG. 16, in the cartridge 1C, when the second handle 84 abuts the second side end 14 of the casing 10C and the first handle 81 protrudes from the first side end 13, the first wall 4C is disposed on the distal side of the insertion path 2, and the insertion path 2 and the cavity 6C communicate with each other. In this state, as illustrated in FIG. 17, similar to the foregoing embodiment, when the operation wire 102 and the sheath 101 with which the coupling member 230 after ligation is engaged are inserted through the insertion port 21 and the operation wire 102 is advanced and retracted, the coupling member 230 is removed from the operation wire 102.

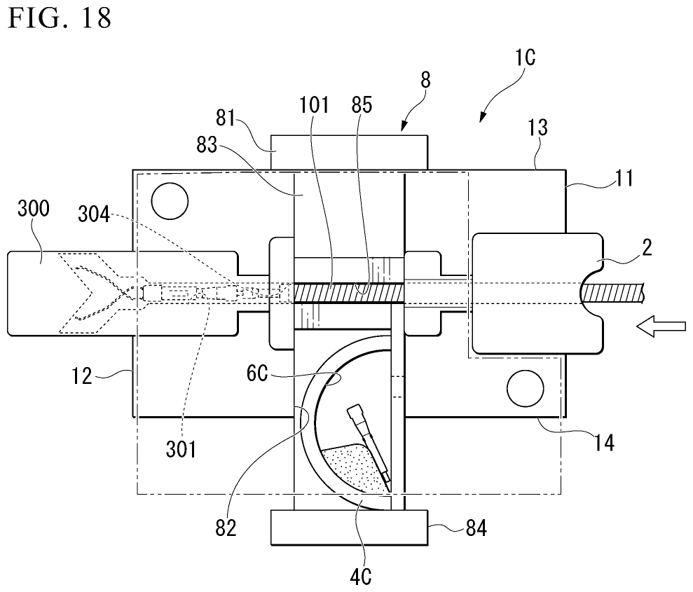

As illustrated in FIG. 18, in the cartridge 1C, when the first handle 81 abuts the first side end 13 of the casing 10C and the second handle 84 protrudes from the second side end 14, the sheath insertion path 85 is disposed on the distal side of the insertion path 2, and the sheath insertion path 85 and the proximal opening 304 communicate with the insertion path 2. When the ligature tool 200 is coupled to the operation wire 102, the sheath 101 is further pushed so that the sheath 101 is inserted to the sheath insertion path 85 and the proximal opening 304. Thereafter, the operation wire 102 is advanced to the accommodation portion 302, and the connector 103 is coupled to the coupling member 230. When the sheath is caused to retract thereafter, the ligature tool 200 becomes in a usable state.

According to the cartridge 1C of the present modified example, similar to the cartridge 1B according to the second modified example, work of removing the coupling member 230 from the operation wire 102 and work of attaching a new ligature tool 200 to the operation wire 102 can be continuously and smoothly performed with one cartridge.

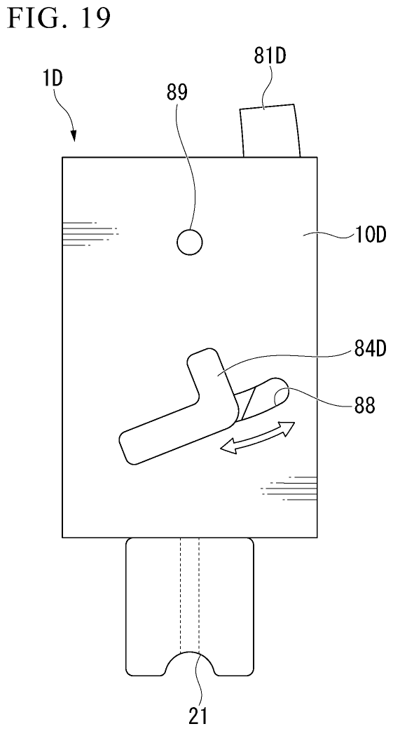

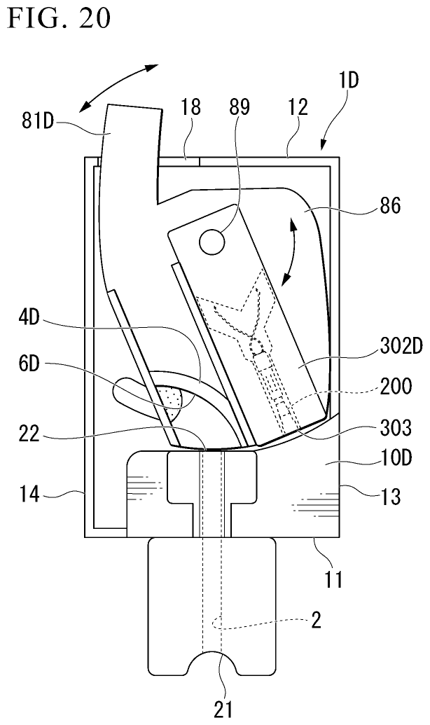

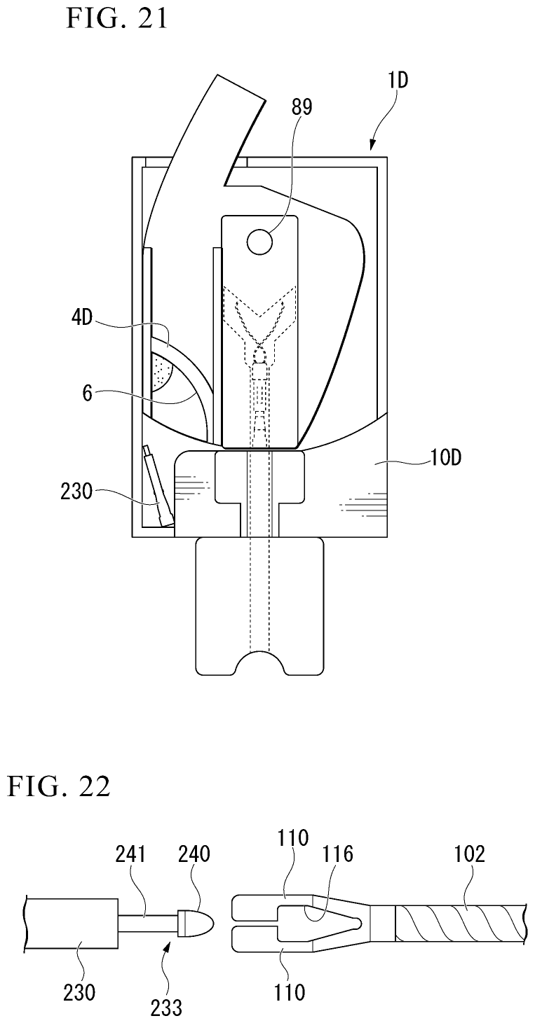

FIGS. 19 to 21 illustrate a cartridge 1D according to a fourth modified example of the exemplary embodiment. In the cartridge 1D according to the present modified example, similar to the second modified example, the detachment cartridge of the coupling member 230 and the attachment cartridge of the ligature tool 200 are integrally provided. FIG. 19 illustrates an appearance of the cartridge 1D. FIGS. 20 and 21 illustrate a partial cross-sectional view of the casing 10D. In the cartridge 1D according to the present modified example, an accommodation portion 302D of the ligature tool 200 and a cavity 6D are formed in a base plate 86. The base plate 86 is coupled to the casing 10D by a shaft 89, and the base plate 86 is configured to be rotatable around the shaft 89. As illustrated in FIG. 19, an arc-shaped slit 88 centering on the shaft 89 is formed in the casing 10D. A first handle 81D is provided in the base plate 86. The first handle 81D protrudes to the outside from the distal end of the casing 10D. The first handle 81D is attached so as to be movable inside the slit 88 formed at the distal end of the casing 10D. A second handle 84D is provided in the base plate 86. The second handle 84D is interlocked with the slit 88 and exposed to the outside of the casing 10D.

The insertion port 21 and the insertion path 2 are provided at the proximal end 11 of the casing 10D. The distal opening 22 communicates with the inside of the casing 10D. An edge end of the base plate 86 turns in the vicinity of the distal opening 22. When a user operates the first handle 81D or the second handle 84D, the sheath insertion portion 303 of the accommodation portion 302 of the ligature tool 200 or the cavity 6D communicates with the distal opening 22. At the time of an operation of removing the coupling member 230, a user moves a first wall 4D by operating the first handle 81D or the second handle 84D. As illustrated in FIG. 20, after the cavity 6D is caused to communicate with the distal opening 22, the sheath 101 is inserted into the insertion port 21. At the time of attachment of the ligature tool 200, a user moves the base plate 86 by operating the first handle 81D or the second handle 84D. As illustrated in FIG. 21, after the sheath insertion portion 303 of the accommodation portion of the ligature tool 200 or the cavity 6D is communicated with the distal opening 22, an operation of attaching the ligature tool 200 is performed. In either case, if the operation wire 102 is advanced and retracted after the sheath 101 is inserted through the insertion port 21, an operation of attaching the ligature tool 200 or an operation of removing the coupling member 230 can be performed.

According to the cartridge 1D of the present modified example, similar to the cartridge 1B according to the second modified example, work of removing the coupling member 230 from the operation wire 102 and work of attaching a new ligature tool 200 to the operation wire 102 can be continuously and smoothly performed with one cartridge. According to the cartridge 1D of the present modified example, work of removing the coupling member 230 and work of attaching the ligature tool 200 can be continuously performed without moving the sheath 101.

In the foregoing embodiments and the modified examples, an example in which the first wall (wall) of the cavity functions as a detachment guide has been described, but the detachment guide is not limited to this constitution. For example, a constitution in which a wall portion serving as a detachment guide is separately provided inside the cavity may be adopted.

In the foregoing embodiments and the modified examples, an example in which the first walls 4, 4C, and 4D are curved has been described, but the first wall (wall) regulating the cavity is not limited to having a curved shape. The first wall need only be constituted to have a surface which is inclined with respect to the longitudinal axis X1 of the operation wire inserted through the insertion port 21. For example, it may be substantially a linearly inclined surface intersecting the longitudinal axis X1.

The ligature tool 200 mentioned in the foregoing embodiments is an example, and the present disclosure may also be applied to the ligature tool 200 having a different form. The form of the clip main body of the ligature tool 200, the holding tube 202, and the like may be applied to the ligature tool 200 having a known form. In the foregoing embodiments, an example in which the hook portion of the coupling member is interlocked with the connection portion of the clip main body has been described, but the form of connecting the clip main body and the coupling member to each other is not limited to this example.

In the foregoing embodiments, an example in which the coupling member of the ligature tool 200 includes a pair of legs and an opening and the arrowhead portion 106 is provided in the connector of the operation wire has been described. However, the foregoing cartridge is not limited to disengagement between the coupling member and the connector of the operation wire in this constitution example and may also be applied to the coupling member having a different form. For example, as in the modified example illustrated in FIG. 22, the present disclosure can also be applied to an example in which a pair of legs 110 and an opening 116 are provided at the distal end of the operation wire 102, the shaft 105 and the arrowhead portion 106 are provided in the proximal engagement portion of the coupling member of the ligature tool 200, and the arrowhead portion 106 is engaged with the opening 116 between the pair of legs 110.

Hereinabove, each of the embodiments of the present disclosure has been described, but the technical scope of the present disclosure is not limited to the foregoing embodiments. Within a range not departing from the gist of the present disclosure, the combination of the constituent elements in each of the embodiments can be changed, and various changes can be added to or deleted from each of the constituent elements. The present disclosure is not limited by the foregoing description and is limited only by the scope of the accompanying claims.

What is claimed is:

1. A cartridge comprising:
an insertion port configured to receive at least a part of an applicator;
a first region configured to accommodate a clip unit that is configured to be releasably coupled to the applicator; and
a second region configured to remove a coupling member from the applicator.

2. The cartridge according to claim 1, wherein
the clip unit includes a clip arm configured to be opened and closed, and
the clip unit includes a second coupling member that is configured to couple the clip unit with the applicator.

3. The cartridge according to claim 2,
wherein the second region is configured to receive the coupling member such that the coupling member is tilted at a detachment angle at which the coupling member is capable of being detached from the applicator.

4. The cartridge according to claim 3, wherein
the second region is a cavity defined by an internal wall of the cartridge,
the second region communicates with the insertion port, and
the second region is configured so that the applicator and the coupling member inserted through the insertion port are movable in the second region.

5. The cartridge according to claim 3, wherein
the second region is a cavity formed in the cartridge, and
a detachment guide is provided inside the cavity and configured to cause the coupling member to tilt at the detachment angle in response to abutment of the coupling member against the detachment guide.

6. The cartridge according to claim 3, wherein
the second region is a cavity formed in the cartridge, and
a retraction restricting guide is provided inside the cavity and configured to restrict retraction of the coupling member when the applicator is retracted.

7. The cartridge according to claim 6,
wherein the retraction restricting guide is provided on a wall defining the cavity, and includes an inclined surface inclined from a distal end portion of the wall toward the insertion port.

8. The cartridge according to claim 3, wherein
the insertion port extends in a longitudinal direction from an opening of the insertion port on a proximal end side of the cartridge toward a distal end side of the cartridge, and the cartridge extends from a first end side to a second end side in a lateral direction orthogonal to the longitudinal direction of the insertion port;
the second region is a cavity formed in the cartridge;
a detachment guide is formed inside the cavity and extends from a first end in a distal direction so as to be curved in a direction intersecting an advance-retract direction of the applicator through the insertion port;
a retraction restricting guide is formed inside the cavity and includes an inclined surface inclined from a second end side of the detachment guide toward the insertion port, the retraction restricting guide being configured to restrict retraction of a distal end of the coupling member which is tilted at the detachment angle when the applicator is retracted; and
a distance between the first end of the detachment guide and a proximal end of the inclined surface of the retraction restricting guide is shorter than a length of the coupling member which has been inserted into the cavity.

9. The cartridge according to claim 3, wherein
the coupling member is configured to be releasably engaged with a distal end portion of a wire of the applicator so as to be moveable in a direction intersecting a longitudinal axis of the wire, and
either (a) or (b) is satisfied:
(a) the coupling member includes an opening through which the wire is configured to extend when the coupling member is tilted at the detachment angle and detached from the wire, or
(b) a distal end portion of the wire includes an opening through the coupling member is configured to extend when the coupling member is tilted at the detachment angle and detached from the wire.

10. The cartridge according to claim 2, wherein
the clip unit is configured to grasp a biological tissue, and
the second coupling member is configured to be separated from the clip unit after the clip unit ligates the biological tissue.

11. The cartridge according to claim 1, wherein
the insertion port includes a first insertion port and a second insertion port,
the first insertion port communicates with the first region, and
the second insertion port communicates with the second region.

12. The cartridge according to claim 1, further comprising:
a casing in which the insertion port is provided; and
a slidable region that includes the first region and the second region and is configured to slide with respect to the insertion port.

13. A method of using a cartridge comprising:
inserting a coupling member and an applicator into a cartridge in a first state in which the coupling member is releasably connected to the applicator;
shifting to a second state in which the coupling member is able to be detached from the applicator by advancing and retracting the applicator; and
detaching the coupling member from the applicator by advancing and retracting the applicator in the second state.

14. The method of using a cartridge according to claim 13, further comprising
connecting a second coupling member to the applicator by using the cartridge in a state in which the coupling member is detached from the applicator.

15. The method of using a cartridge according to claim 14, wherein
the coupling member is detached from the applicator in a state in which the applicator is inserted into a first insertion port of the cartridge, and
the second coupling member is connected to the applicator by retracting the applicator from the first insertion port, and inserting the applicator into a second insertion port of the cartridge.

16. The method of using a cartridge according to claim 13, further comprising
sliding a slider comprised by the cartridge in a state in which the coupling member is detached from the applicator, and
connecting the applicator to a second coupling member.

17. The method of using a cartridge according to claim 13, wherein
the coupling member extends along a longitudinal axis of the applicator in the first state, and
the coupling member extends in a direction intersecting the longitudinal axis in the second state.

18. The method of using a cartridge according to claim 13,
wherein the coupling member and the applicator are shifted to the second state by advancing the applicator inside the cartridge such that the coupling member abuts against an inner wall of the cartridge.

19. The method of using a cartridge according to claim 13,
wherein the coupling member is detached from the applicator by retracting the applicator inside the cartridge such that the coupling member in the second state is caused to abut against the inner wall of the cartridge.

20. The method of using a cartridge according to claim 13,
wherein the applicator includes one or both of a wire and a sheath.
